(19) United States
(12) Patent Application Publication
(10) Pub. No.: US 2022/0313280 A1
(43) Pub. Date: Oct. 6, 2022

(54) Tool Assembly And Methods For Robotic-Assisted Surgery

(71) Applicant: MAKO Surgical Corp., Weston, FL (US)

(72) Inventors: Alvin Sarendranath, Hackensack, NJ (US); Amir A. Sharifi-Mehr, Bloomingdale, NJ (US); Shawn D. Stad, Lakeville S, MA (US); Oliver Buchert, Franklin Lakes, NJ (US); Kornelis Poelstra, Destin, FL (US); Zinoviy Sosnov, Fair Lawn, NJ (US); Matthew J. McGirt, Charlotte, NC (US)

(73) Assignee: MAKO Surgical Corp., Weston, FL (US)

(21) Appl. No.: 17/628,249

(22) PCT Filed: Jul. 17, 2020
(86) PCT No.: PCT/US2020/042487

§ 371 Date: Jan. 19, 2022

Related U.S. Application Data
(60) Provisional application No. 62/876,329, filed on Jul. 19, 2019.

(51) Int. Cl. A61B 17/16; A61B 17/17; A61B 34/30

(52) U.S. Cl. CPC A61B 17/1633; A61B 17/1757; A61B 17/1671; A61B 34/30; A61B 17/1631; A61B 2017/00991

(57) ABSTRACT

Tool assemblies and methods are disclosed, such as for robotic-assisted surgery with a robot. The tool assembly includes a working tool (44) sized to be coaxially movable within the dilator (60). A locking mechanism (70) releasably couples the working tool (44) and the dilator (60) to one another such that, in a locked configuration, the robot supports the tool assembly. An actuator (86) is configured to receive an input to move the locking mechanism to an unlocked configuration and permit axial movement of the working tool within the dilator. The dilator (60) may include at least one spike (104) disposed within the grooves (128) to penetrate bone. The dilator (60) may include an inner sleeve and outer sleeve coaxially movable relative to one another to move the tool assembly from an initial configuration to a deployed configuration in which the spikes are exposed beyond the sleeve(s). Methods of preparing a pedicle of the spine with the tool assembly with robotic assistance are also disclosed.

### CROSS-REFERENCE TO RELATED APPLICATIONS

The subject application claims priority to and all the benefits of U.S. Provisional Patent Application No. 62/876,329, filed Jul. 19, 2019, the contents of which are hereby incorporated by reference in its entirety.

### BACKGROUND

Robotic-assisted surgery facilities precise placement of instrumentation in a desired position and orientation relative to anatomy of interest. One example includes creating a pilot hole for the placement of a screw within a pedicle of a vertebra during a spinal fusion procedure. While the robot ensures the desired pose of the working tool, it is also necessary to establish a working channel through overlying tissue to the underlying bony anatomy. In conventional minimally invasive surgery, a series of tubular dilators are sequentially inserted with the last-placed dilator (e.g., a retractor) providing the working channel. With the working tool coupled to and supported by the robot in robotic-assisted surgery, misalignment of the manually-placed dilator with the pose of the working tool may prevent satisfactory passage of the working tool through the dilator, for example, without undue trauma to the surrounding tissue. Such an arrangement further requires the surgeon cooperatively manipulate each of the dilator and the robot to ensure satisfactory passage of the working tool through the dilator. Another arrangement in the context of robotic-assisted surgery includes the robot supporting the last-placed dilator as a guide tube, and the guide tube constrains the working tool and other instrumentation directed therethrough by the surgeon. However, positioning and/or operation of the working tool itself is a manual process, which is not robotically assisted.

Furthermore, in the context of preparing a pedicle for screw insertion, rotation of the cutting tool may result in entrapment of the nearby soft tissue, which may further bias against the trajectory of the cutting tool. These biasing forces can negatively contribute to incorrect location of the pilot hole and eventually incorrect screw placement, or even worse potential complications. Therefore, there is a need in the art for an improved tool assembly and methods for robotic-assisted surgery.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In one aspect, a tool assembly is disclosed. The tool assembly includes a dilator having a sleeve defining a lumen, and a working tool. The working tool includes a shank configured to be coupled to and supported by a device, and a cutting member coupled to the shank. The working tool is sized to be coaxially movable within the lumen of the dilator. The tool assembly includes a locking mechanism having first locking feature coupled to the shank, and a second locking feature coupled to the sleeve. The first and second locking features are configured to releasably engage one another to prevent axial movement of the working tool relative to the dilator such that the working tool supports the dilator when the working portion is attached to the device. The locking mechanism further includes an actuator configured to receive an input to disengage the first and second locking features and permit axial movement of the working tool within the lumen of the dilator.

In one implementation, the tool assembly is for robotic-assisted surgery with a robot. The shank is configured to be coupled to and supported by the robot. The first and second locking features are configured to releasably engage one another to prevent axial movement of the working tool relative to the dilator such that the working tool supports the dilator when the working tool is coupled to the robot. The robot can be provided on a mobile cart, attached to a surgical table, attached to a gantry or imaging device, or mounted to the patient. The robot can also be implemented as a hand-held device configured to be held and supported by the hand of the user against the force of gravity.

In one implementation, the cutting member can be any of the following: a bur head, a bur, sharpened flutes for a drill or a tap drill, a point or a blunted end for a probe, a screw head for a screwdriver, a blade for a scalpel, among others.

In some implementations, the actuator is any device configured to disengage the first and second locking features. In one implementation, the actuator is a mechanical actuator. The actuator may also be electro-mechanical, magnetic, passively actuated, actively actuated, a push-button, release lever, push tab, a biasing mechanism, a rotational member, a linearly translating member, or the like. In some implementations, the actuator can be further configured to releasably engage the first and second locking features.

In some implementations, the dilator further comprises at least one spike. The at least one spike can be coupled to the sleeve and configured to penetrate tissue, such as bone.

In some implementations, the sleeve is an inner sleeve defining the lumen. In some implementations, the dilator further comprising an outer sleeve coaxially disposed over the inner sleeve. In some implementations, the inner sleeve is movably coupled to the outer sleeve. In other configurations, the outer sleeve is movably coupled to the inner sleeve.

In some implementations, the inner sleeve has an inner sleeve length defined between the second locking feature and a distal end of the inner sleeve, and the outer sleeve has an outer sleeve length defined between opposing proximal and distal ends of the outer sleeve. In some implementations, the inner sleeve length is greater than the outer sleeve length. In some implementations, the inner sleeve length is less than the outer sleeve length. In some implementations, the inner sleeve length is equal to the outer sleeve length.

In some implementations, the working tool has a tool length configured to align the cutting member to the distal end of the inner sleeve when the first and second locking features are engaged.

In some implementations, the outer sleeve further comprises a dilator tip defining a distal end of the outer sleeve. In some implementations, the dilator tip is at least partially formed from a resilient material. In some implementations, the dilator tip is configured to expand over the inner sleeve as the outer sleeve is retracted relative to the inner sleeve.

In some implementations, the at least one spike is movably coupled to the sleeve. In some implementations, the at least one spike is configured to be selectively extended beyond a distal end of the sleeve to penetrate the bone. In some implementations, the at least one spike further comprises an elongate spike body slidably disposed within a groove defined by the sleeve. In some implementations, the dilator further comprises a biasing element operably coupled to the at least one spike and configured to selectively extend the at least one spike beyond the distal end of the sleeve to penetrate the bone.

In some implementations, the dilator further comprises a retention mechanism releasably coupling the sleeve and the at least one spike. In some implementations, the retention mechanism is configured to receive an input to permit movement of the at least one spike relative to the sleeve. In some implementations, the biasing element is a coil spring disposed about the sleeve between the locking mechanism and the retention mechanism. In other implementations, the biasing element is another type of spring, such as a compression, extension, torsion, leaf spring or the like. The biasing element can also be a resilient material.

In some implementations, the first locking feature comprises a necked portion of the shank defining two stepped surfaces. In some implementations, the second locking feature comprises a biasing element coupled to the actuator and configured to urge the actuator into releasable engagement with one of the stepped surfaces.

In a second aspect, a method is provided for performing surgery with a device, a tool assembly including a dilator, a working tool, and a locking mechanism releasably coupling the dilator and the working tool. With the working tool is coaxially disposed within the dilator and with the locking mechanism in a locked configuration in which axial movement of the working tool relative to the dilator is prevented, the working tool is coupled to the device such that the device supports the working tool and the dilator. The device is operated to advance a distal end of the working tool and a distal end of the dilator into an incision in overlying tissue and above or in engagement with a bone. The locking mechanism is actuated from the locked configuration to an unlocked configuration in which axial movement of the working tool relative to the dilator is permitted. The device is operated to advance the distal end of the working tool and to manipulate the bone, wherein a position of the dilator is maintained by the overlying tissue supporting the dilator.

In one implementation, the method is for performing robotic-assisted surgery with a robot. The robot can be provided on a mobile cart, attached to a surgical table, attached to a gantry or imaging device, or mounted to the patient. The robot can also be implemented as a hand-held device configured to be held and supported by the hand of the user against the force of gravity.

In one implementation, the dilator includes at least one spike. In one implementation, the method further comprises facilitating the engagement of the at least one spike with the bone. In one implementation, the step of facilitating the engagement of the at least one spike with the bone is after the step of actuating the locking mechanism from the locked configuration to the unlocked configuration.

In one implementation, the dilator further includes an inner sleeve including the at least one spike, and an outer sleeve coaxially disposed over the inner sleeve. In one implementation, the method further comprises retracting the outer sleeve relative to the inner sleeve to expose the at least one spike. In one implementation, the inner sleeve and the outer sleeve include complementary threads. In one implementation, the method comprises providing input to the outer sleeve to retract the outer sleeve relative to the inner sleeve, or vice versa, to expose the at least one spike. In one example, the input is a rotational input, but may be other types of input, such as linear or translational input.

In one implementation, the dilator comprises a sleeve. In one implementation, the sleeve defines a groove and the at least one spike is disposed within the groove. In one implementation, the method includes advancing the at least one spike beyond a distal end of the sleeve. In one implementation, the dilator further includes a collar coupled to the at least one spike. In one implementation, the method includes impacting the collar with an impacting device to advance the at least one spike beyond the distal end of the sleeve.

In one implementation, the tool assembly further includes a retention mechanism releasably coupling the at least one spike and the dilator. In one implementation, the method includes providing an input to the retention mechanism to disengage the at least one spike from the dilator.

One example of a tool assembly is disclosed. The tool assembly includes a dilator and a working tool. The dilator includes an inner sleeve defining a lumen and having a spike, and an outer sleeve coaxially disposed over the inner sleeve. At least one of the inner and outer sleeves is movable from an initial configuration in which the spike is recessed within the outer sleeve, and a deployed configuration in which the spike extends beyond the outer sleeve for penetrating bone. The working tool includes a shank configured to be coupled to and supported by a device, and a cutting member coupled to the shank. The working tool sized to be slidably and coaxially movable within the lumen of the dilator. The tool assembly includes a locking mechanism releasably coupling the working tool and the dilator.

In one implementation, the tool assembly is for robotic-assisted surgery with a robot. The shank is configured to be coupled to and supported by the robot. The first and second locking features are configured to releasably engage one another to prevent axial movement of the working tool relative to the dilator such that the working tool supports the dilator when the working tool is coupled to the robot. The robot can be provided on a mobile cart, attached to a surgical table, attached to a gantry or imaging device, or mounted to the patient. The robot can also be implemented as a hand-held device configured to be held and supported by the hand of the user against the force of gravity.

In some implementations, the locking mechanism further comprises a necked portion of the shank defining two stepped surfaces. In some implementations, an actuator is positioned for releasable engagement with one of the stepped surfaces.

In some implementations, the inner sleeve has an inner sleeve length defined between opposing proximal and distal ends of the inner sleeve, and the outer sleeve has an outer sleeve length defined between opposing proximal and distal ends of the outer sleeve, wherein the inner sleeve length is greater than the outer sleeve length. In some implementations, the inner sleeve length is less than the outer sleeve length. In some implementations, the inner sleeve length is equal to the outer sleeve length.

In some implementations, the outer sleeve further comprises a dilator tip. In some implementations, the dilator tip defines the distal end of the outer sleeve. In some implementations, the dilator tip is at least partially formed from resilient materials. In some implementations, the dilator tip is configured to expand over the inner sleeve as the outer sleeve is moved from the initial configuration to the deployed configuration.

Any of the above aspects can be combined in full or in part. Any features of the above aspects can be combined in full or in part. Any of the above implementations for any aspect can be combined with any other aspect. Any of the above implementations can be combined with any other implementation whether for the same aspect or different aspect.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a robotic surgical system.

FIG. 2 is a perspective view of a tool assembly supported by a robot above a representation of the spine.

FIG. 3 is a side elevation view of a tool assembly.

FIG. 4 is a sectional view of the tool assembly of FIG. 3.

FIG. 5 is a perspective view of a tool assembly supported by a robot above a representation of the spine. The tool assembly is in an initial configuration.

FIG. 5A is a detailed view of FIG. 5 within box 5A.

FIG. 6 is a perspective view of the tool assembly of FIG. 5 in a deployed configuration.

FIG. 6A is a detailed view of FIG. 6 within box 6A.

FIG. 7 is a side elevation view of the tool assembly of FIGS. 5 and 6 with the tool assembly in the initial configuration.

FIG. 8 is a side elevation view of the tool assembly of FIGS. 5 and 6 with the tool assembly in the deployed configuration.

FIG. 9 is a sectional view of the tool assembly of FIGS. 5 and 6 with the tool assembly in the deployed configuration.

FIG. 10 is a side elevation view of a tool assembly in the initial configuration.

FIG. 11 is a side elevation view of the tool assembly of FIG. 10 with the tool assembly in the deployed configuration.

FIG. 12 is a sectional view of the tool assembly of FIG. 10 in the deployed configuration.

FIG. 13 is a perspective view of a tool assembly supported by a robot above a representation of the spine. The tool assembly is in an initial configuration.

FIG. 13A is a detailed view of FIG. 13 within box 13A.

FIG. 14 is a perspective view of the tool assembly of FIG. 13 in the deployed configuration.

FIG. 14A is a detailed view of FIG. 14 within box 14A.

FIG. 15 is a side elevation view of a tool assembly in the initial configuration.

FIG. 16 is a side elevation view of the tool assembly of FIG. 15 in the deployed configuration.

FIG. 17 is a sectional view of the tool assembly of FIG. 15 in the deployed configuration.

FIG. 18 is a perspective view of a tool assembly in the initial configuration.

FIG. 19 is a side elevation view of the tool assembly of FIG. 18 in the deployed configuration.

FIG. 20 is a sectional view of the tool assembly of FIG. 18 in the deployed configuration.

FIG. 21 is a perspective view of a retention mechanism.

FIG. 22 is a sectional view of the tool assembly of FIG. 18 in the initial configuration. The retention mechanism is in a first position.

FIG. 23 is a sectional view of the tool assembly of FIG. 18 in the deployed configuration. The retention mechanism is in a second position.

FIG. 24 is an elevation view of a locking mechanism of the tool assembly.

FIG. 25 is a portion of a shank of a working tool of the tool assembly. The portion of the shank includes a locking feature of the locking mechanism.

FIG. 26 is a plan view of the locking mechanism in an engaged configuration. The portion of the shank is shown in section.

### DETAILED DESCRIPTION

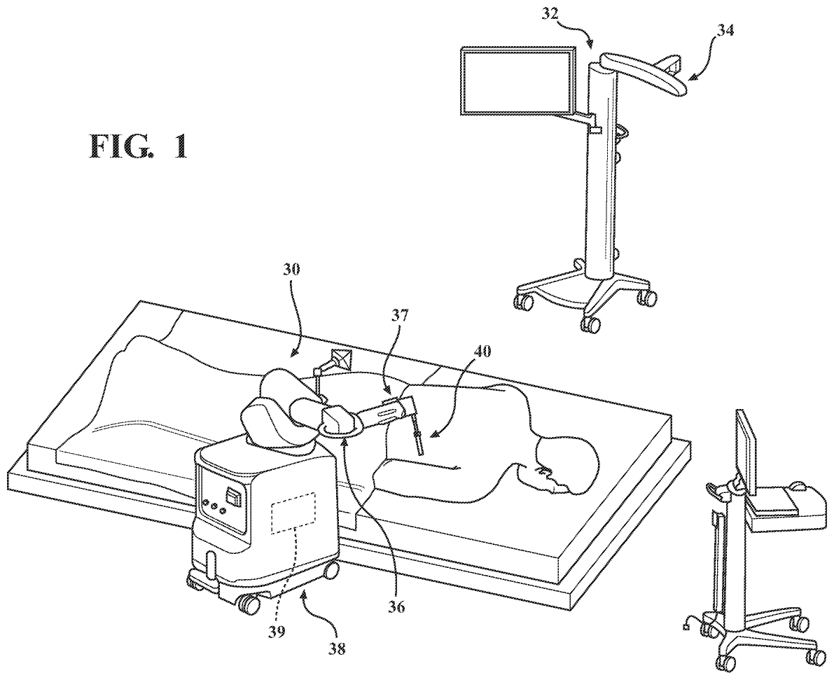

FIG. 1 shows a robotic system 30 with a tool assembly 40 operable to surgically manipulate tissue of a patient. The robotic system 30 may be used with the tool assembly 40 for various methods and procedures in which the tissue (e.g., bone underlying or adjacent soft tissue or other tissue) is prepared for implantation of screws or for placement of other types of implants. Of particular interest is a spinal procedure in which the pedicle(s) of one or more vertebrae are prepared for implantation of a pedicle screw. The robotic system 30 includes a navigation system 32 with a localizer 34, and a robotic manipulator (e.g., a robotic arm 36 mounted to a base 38). In one example, the robotic manipulator is the Robotic Interactive Orthopedic (RIO™) System manufactured by MAKO Surgical Corp. (Florida). The tool assembly 40 is coupled to the robotic arm 36, and the tool assembly 40 may assume any number of permutations to be described throughout the present disclosure. The robotic system 30 may have configurations other than that shown in FIG. 1.

The robotic system 30 may include an end effector 37. The end effector 37 is configured to be removably coupled to the tool assembly 40. In one implementation, the end effector 37 includes a hex nut and a collet, and the hex nut compresses the collet to facilitate attachment of the tool assembly 40 to the end effector 37. Further, the end effector 37 may be configured to provide a torque to impart rotational movement to a working tool 44 of the tool assembly 40. Additionally, or alternatively, the movement imparted by the end effector 37 may be translational such that the tool assembly 40 is capable of moving in six or more degrees of freedom.

A robotic controller 39 (schematically shown in phantom) may be configured to control or constrain the end effector 37 and/or the tool assembly 40 as the surgeon facilitates movement of the tool assembly 40. For example, the robotic controller 39 may be configured to control the robotic arm 36 with actuators (e.g., joint motors) to provide haptic feedback to the surgeon via the robotic arm 36. The haptic feedback helps to inhibit the surgeon from manually moving the tool assembly 40 beyond predefined virtual boundaries associated with the surgical procedure. One example of a haptic feedback system with associated haptic objects defining virtual boundaries is described in commonly owned U.S. Pat. No. 8,010,180, issued Aug. 30, 2011, the entire disclosure of which hereby incorporated by reference. The robotic controller 39 and robotic manipulator 30 may control the end effector 47 and any of the associated components attached thereto relative to the target site according to methods such those described in US Patent Application Publication No. US2019/0090966A1, the entire contents of which are hereby incorporated by reference in their entirety. The robotic controller 39 may operate the robotic manipulator 30 according to various other techniques not specifically described herein.

The working tool 44 may be a bur, a drill, a tap drill, a probe, a screwdriver, and a scalpel, among others. More generally, the working tool 44 may be any elongate instrument configured to manipulate tissue. With reference to FIGS. 4, 9, 12, 17 and 20, the working tool 44 includes a shank 46, and a working portion 48 coupled to the shank 46. In one convention, the shank 46 may be considered a proximal region of the working tool 44, and the working portion 48 may be considered a distal region of the working tool 44. In other words, the shank 46 may define a proximal end (not identified) of the working tool 44, and the working portion 48 may define a distal end 50 opposite the proximal end. The proximal end of the shank 46 may be operably coupled to the end effector 47 of the robotic system 30. Further, with the shank 46 operably coupled to the end effector 47, the working tool 44 (and a dilator 60 to be described) may be supported by the robotic system 30. In other words, owing to the architecture of the robotic system 30, the tool assembly 40 may be supported, suspended, or otherwise held above the patient without requiring separate or independent support or manipulation from the surgeon (see FIG. 1).

As best shown in the sectional views of FIGS. 4, 9, 12, 17 and 20, a transition 52 from the shank 46 to the working portion 48 may be associated with a taper or narrowing of the outer diameter of the working tool 44. The shank 46 may have an outer diameter approximately sized to an inner diameter of the dilator 60 such that the shank 46 is snugly, slidably, coaxially, and rotatably disposed within the dilator 60. The working portion 48 may have an outer diameter smaller than the outer diameter of the shank 46 such that an annular gap exists between the working portion 48 and the dilator 60. Other suitable geometries of the working tool 44 are contemplated, for example, such as the geometry shown in FIG. 4 in which two transitions 52 are present.

The working portion 48 may include a cutting member 54 defining the distal end 50 of the working tool 44. In the implementation depicted, the cutting member 54 may be a bur head such that the working tool 44 may be considered a bur. Alternatively, the cutting member 54 may be sharpened flutes for a drill or a tap drill, a point or a blunted end for a probe, a screw head for a screwdriver, a blade for a scalpel, among others.

The dilator 60 includes a sleeve 62 defining a lumen 64. The sleeve 62 may be an elongate tubular structure with a length defined between a proximal end 66 opposite a distal end 68 of the sleeve 62. The sleeve 62 is shown as having a substantially constant inner diameter, outer diameter, and thickness between the proximal and distal ends 66, 68. Other suitable geometries are contemplated, for example, the inner and outer diameters may increase away from the distal end 68 such that the sleeve 62 has a tapering in profile. A tapering profile may be particularly well suited for dilating the tissue as the dilator 60 is directed through the overlying tissue. The thickness of the sleeve 62 may be constant or vary.

The working tool 44 is configured to be snugly, slidably, coaxially, and rotatably movable within the lumen 64 of the dilator 60. At certain points during a procedure, it may be desirable to have the dilator 60 axially fixed relative to the working tool 44, and at other points during the procedure to have the dilator 60 be axially movable relative to the working tool 44. For example, as the tool assembly 40 is initially placed through an incision in the overlying tissue towards the underlying bone, it may be desirable to have the sleeve 62 axially fixed relative to the working tool 44 such that the sleeve 62 dilates the overlying tissue and establishes a working channel for the working tool 44. After the sleeve 62 is positioned above and/or engaging the bone in manners to be described, it may be desirable for the working tool 44 to be axially movable relative to the sleeve 62.

The resiliency of the overlying tissue may laterally support the sleeve 62, and the working tool 44 may be advanced relative to the dilator 60 to resect the bone. The tool assembly 40 advantageously accommodates the aforementioned considerations with a locking mechanism 70. The locking mechanism 70 is configured to releasably couple and decouple the working tool 44 and the dilator 60. With the locking mechanism 70 in a locked configuration in which the working tool 44 and the dilator 60 are releasably coupled, and with the shank 46 operably coupled to the end effector 47, the working tool 44 and the dilator 60 may be supported by the robotic system 30. The resulting configuration is best shown in FIGS. 1 and 2, 5, 6 and 14 with the tool assembly 40 effectively suspended above the spine (S) of the patient. As a result, with the locking mechanism 70 in the locked configuration, the robotic controller 39 may control or constrain the position and/or orientation of both of the working tool 44 and the dilator 60 as the tool assembly 40 is initially placed through an incision in the overlying tissue towards the underlying bone, thereby ensuring optimal placement of the dilator 60. Once the tool assembly 40 is in a desired position, for example, on a trajectory to prepare a pedicle of a vertebra, the locking mechanism 70 may be moved from the locked configuration to an unlocked configuration in which the working tool 44 is movable relative to the dilator 60. The robotic controller 39 may control or constrain the position and/or orientation of the working tool 44, and the position and/or orientation of the dilator 60 may be at least substantially maintained by engagement with the bone and/or support from adjacent overlying tissue.

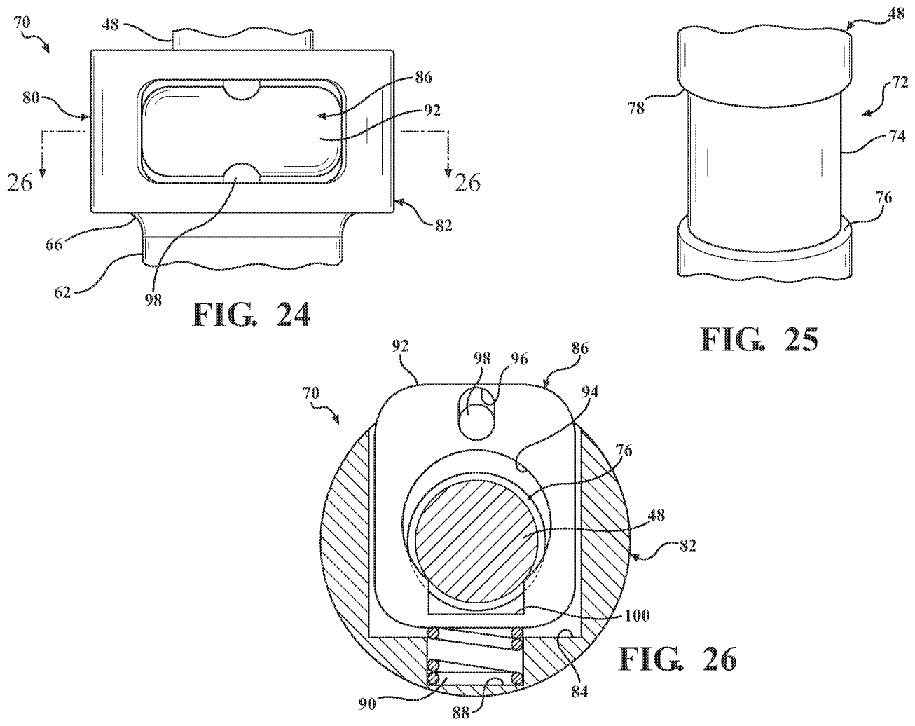

With concurrent reference to FIGS. 24-26, one implementation of the locking mechanism 70 is shown. The locking mechanism 70 may include a first locking feature 72 coupled to the working tool 44. FIG. 25 shows the first locking feature 72 coupled to and/or forming a part of the shank 46 of the working tool 44. The first locking feature 72 may include a necked portion 74 of the shank 46 such that an indented or stepped surface 76 is formed between adjacent portions of the shank 46. More particularly, the shank 48 has an outer diameter, and the necked portion 74 has an outer diameter that is smaller than the outer diameter of the shank 46. The result is the first or lower stepped surface 76, and a second or upper stepped surface 78 may also be formed.

As shown in FIG. 24, for example, the locking mechanism 70 may include a second locking feature 80 coupled to the dilator 60, and more particularly to the sleeve 62 of the dilator 60. The second locking feature 80 is configured to releasably engage the first locking feature 72. The second locking feature 80 may include a collar 82 coupled to the sleeve 62 of the dilator 60.

As best shown in FIGS. 4, 9, 12, 17 and 20, the collar 82 may be a widened aspect of the sleeve 62 at its proximal end 66. The collar 82 is shown as being formed integrally with the sleeve 62, however, the collar 82 and the sleeve 62 may be discrete components coupled to one another. FIG. 26 shows a sectional view of the locking mechanism 70 taken axially through the collar 82 (and the shank 46). The collar 82 defines a recess 84 sized to movably receive an actuator 86 and may further define a secondary recess 88 sized to receive a biasing element 90. The actuator 86 may akin to a button and may include a control surface 92 for receiving an input from a user. The actuator 86 may define a bore 94 sized to removably receive the shank 46 of the working tool 44, and a slot 100 in communication with the bore 94. The actuator 86 may define an aperture 96 sized to movably receive a locking pin 98 of the locking mechanism 70. The biasing element 90 is disposed within the secondary recess 88 in a compressed state such that the biasing element 90 provides a force on the actuator 86 towards the control surface 92. The locking pin 98 limits the movement of the actuator 86 relative to the collar 82 (e.g. being ejected from the recess 84 subsequent to the working tool 44 being removed) while permitting movement of the actuator 86 with the input provided to the control surface 92.

FIGS. 24 and 26 may be considered to show the locking mechanism 70 in the locked configuration such that the working tool 44 and the dilator 60 are engaged. The actuator 86 is axially aligned with the necked portion 74, and the biasing element 90 urges an inner surface of the slot 100 into engagement with the necked portion 74 of the shank 46. In other words, the biasing element 90 urges the bore 94 out of coaxial alignment with the shank 46. A thickness of the actuator 86 (see FIG. 24) is less than a length of the necked portion 74 of the shank 46 (see FIG. 25). Owing to the relative thickness of the actuator 86 to the length of the necked portion 74, the slot 100 is positioned between and adjacent the lower and upper stepped surfaces 76, 78. As a result, there is interference engagement between the actuator 86 and the lower and upper stepped surfaces 76, 78, thereby preventing axial movement of the working tool 44 relative to the dilator 60. It is also contemplated that the first and second locking features 72, 80 may be reversed; i.e., the first locking feature 72 being associated with the dilator 60 and the second locking feature 80 being associated with the working tool 44.

An input is provided to the control surface 92 of the actuator 86 to move the locking mechanism 70 from the locked configuration to an unlocked configuration. The input urges the actuator 86 further within the recess 84 of the collar 82 against the force from the biasing element 90. The extent of the movement may be limited by an amount in which the locking pin 98 is permitted to move within the aperture 96. The extent of the movement is at least sufficient to have the slot 100 beyond the outer diameter of the shank 46 (i.e., the slot 100 clears the lower and upper stepped surfaces 76, 78). With the interference engagement between the actuator 86 and the lower and upper stepped surfaces 76, 78 no longer present, the axial movement of the working tool 44 relative to the dilator 60 is permitted. The geometry of the actuator 86 is designed such that no contact occurs between the actuator 86 and the shank 46 of the working tool 44 once the locking mechanism 70 is in the unlocked configuration. In other words, once in the unlocked configuration, the working tool 44 may be rotated—often at high speeds—without contact between the sleeve 62 of the dilator 60 and the shank 46 of the working tool 44.

In certain configurations, the working tool 44 may have a length such that the cutting member 54 aligned with the distal end 68 of the sleeve 62, in the axial direction, when the locking mechanism 70 is in the locked configuration. More particularly and with reference to FIGS. 4, 9, 12, 17 and 20, the length of the working tool 44 may be defined between the first locking feature 72 on the shank 46, for example, the lower stepped surface 76, and the tip of the cutting member 54. The sleeve 62 of the dilator 60 may have a length defined between the second locking feature 80, for example, the proximal end 66, 122 and the distal end 68, 120. The relative lengths of the dilator 60 and the working tool 44 may be configured such that the cutting member 54 is slightly recessed (see FIG. 20), aligned with (see FIG. 4), or slightly protruding (see FIG. 12) from the sleeve 62 with the locking mechanism 70 in the locked configuration. Other relative positioning is contemplated. Alignment of the cutting member 54 and the distal end of the dilator 60 in the axial direction may prevent undesirable ingress of tissue within the lumen 64 of the dilator 60 during advancement of the tool assembly 40 through the overlying tissue.

A workflow of the tool assembly 40 will now be described with reference to FIG. 1 and in the context of preparing a pedicle to receive a pedicle screw. The working tool 44 and the dilator 60 are releasably engaged. The tool assembly 40 may be pre-packaged as such, or the step of releasably engaging the working tool 44 and the dilator 60 may be performed in the surgical suite. For the latter, the working tool 44 may be directed through the lumen 64 of the sleeve 62. The necked portion 74 of the working tool 44 reaches the axial location of the actuator 86, and the actuator 86 is biased by the biasing element 90 into engagement with the necked portion 74. The locking mechanism 70 is in the locked configuration.

The shank 46 of the working tool 44 is coupled to the end effector 37 of the robotic arm 36. The end effector 37 supports both of the working tool 44 and the dilator 60. More particularly, the end effector 37 supports the working tool 44, and the working tool 44 supports the dilator 60. The tool assembly 40 is directed through an incision in the overlying tissue (not shown) with the tool assembly 40 being constrained by the end effector 37. The distal end 68 of the sleeve 62 is directed through the incision and dilates the tissue as the tool assembly 40 is directed downward towards the pedicle of interest. The dilation of the tissue may be associated with a force on the dilator 60 opposite the direction of advancement owing to friction and the resiliency of the overlying tissue. The locking mechanism 70 in the locked configuration (i.e., interference between the actuator 86 and the upper stepped surface 78) prevents axial movement of the dilator 60 relative to the working tool 44, and thus prevents premature exposure of the cutting member 54 of the working tool 44 beyond the distal end 68 of the sleeve 62. The distal end 68 of the sleeve 62 may be positioned just above the bone, for example, two to five millimeters above the pedicle. This distance, again, is based on the constraints from the end effector 37 (e.g., a haptic floor).

The locking mechanism 70 is moved from the locked configuration to the unlocked configuration in the manner previously described. The user provides the input to the control surface 92. The first and second locking features 72, 80 disengage, and axial movement of the working tool 44 relative to the dilator 60 is permitted. In the unlocked configuration, the distal end 68 of the sleeve 62 may remain at least substantially in its position by the forces from the overlying tissue. The robotic controller 39 may control or constrain the position and/or orientation of the working tool 44, and the cutting member 54 of the working tool 44 is advanced distally beyond the distal end 68 of the sleeve 62. The cutting member 54 engages the underlying bone, and the end effector 37 may rotate the cutting member 54, for example, to resect the bone on the desired trajectory and form the pilot hole. The working tool 44 may be removed from the dilator 60, and the working tool 44 may be decoupled from the end effector 37. Another working tool (e.g., a screwdriver with a pedicle screw coupled thereto) may be coupled to the end effector 37, and the screwdriver with pedicle screw may be directed through the lumen 64 of the dilator 60 towards the pilot hole previously formed. Again, the robotic controller 39 may constrain the screwdriver as it is advanced through the sleeve 62 of the dilator 60 and may further precisely control the speed and torque characteristics as the pedicle screw is inserted into and engaging the pedicle. In an alternative implementation, with the pilot hole confidently on the desired trajectory such that the likelihood of misalignment of the pedicle screw is minimal, the surgeon may manually place the pedicle screw through the dilator 60 using conventional instrumentation and techniques.

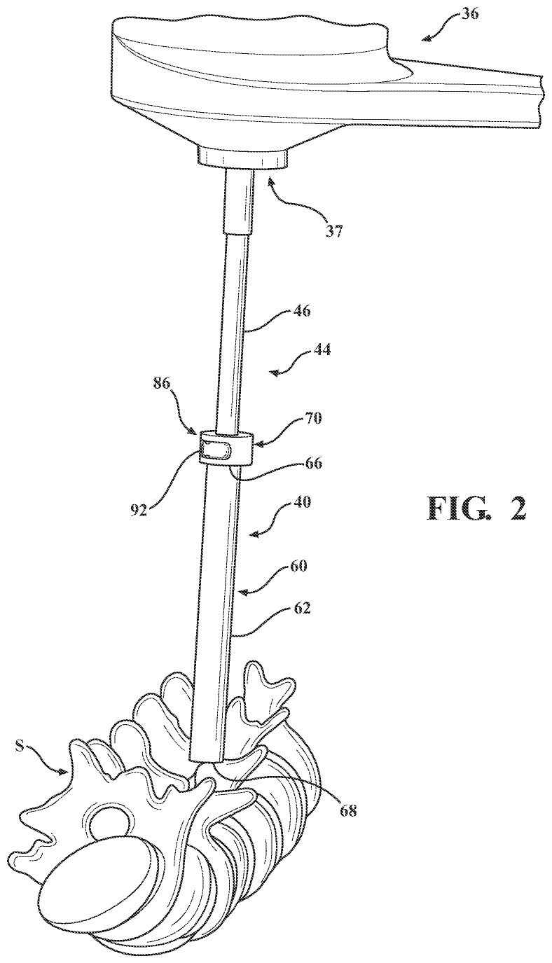

In the implementation shown in FIG. 2, the distal end 68 of the sleeve 62 is blunted and positioned adjacent or above the pedicle. The working channel provided through the overlying tissue to near the underlying bone reduces or eliminates tissue entrapment while facilitating correct placement of the pilot hole. Elimination of tissue entrapment and correct placement of the pilot hole may be further facilitated by engaging the pedicle with the tool assembly 40.

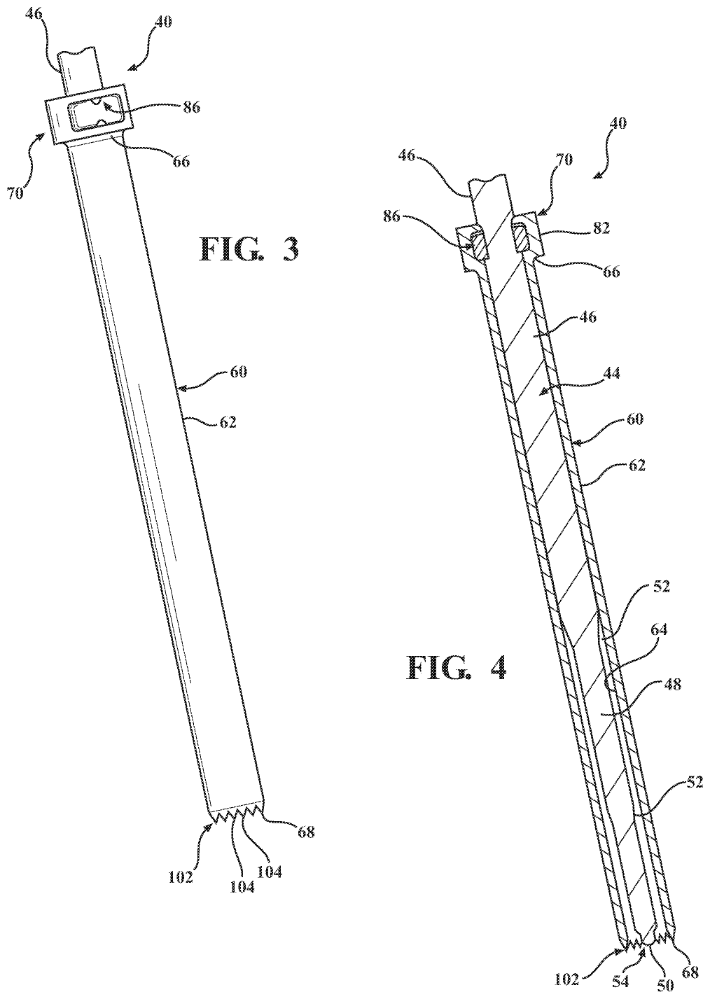

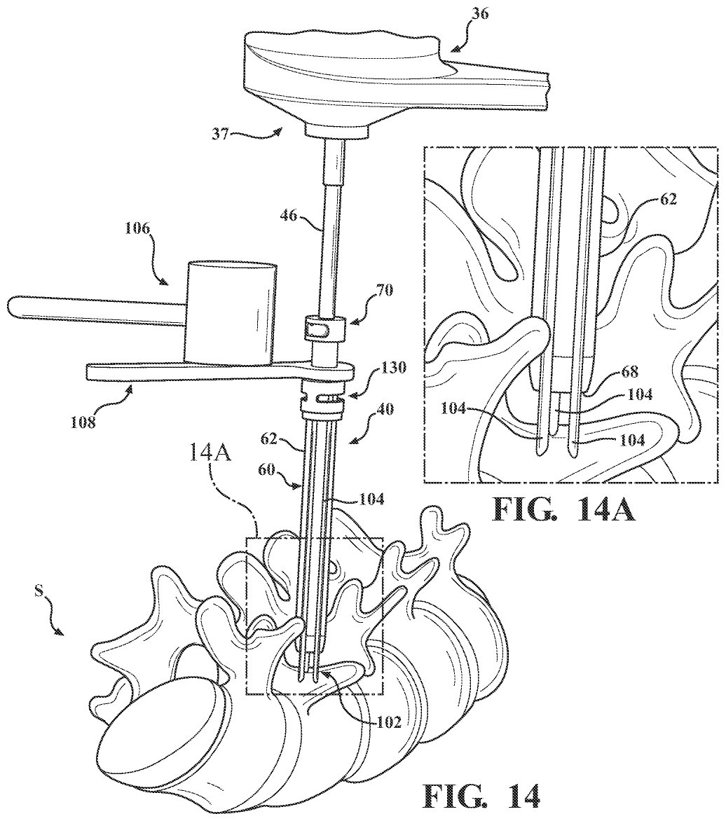

With reference to FIGS. 3 and 4, the dilator 60 of the tool assembly 40 may include an engagement feature 102, for example, at least one spike 104. The spikes 104 may be integral with the sleeve 62 and define the distal end 68 of the dilator 60. The spikes 104 are sufficiently sharp to engage bone. Thus, the tool assembly 40 is directed through an incision in the overlying tissue (not shown) with the tool assembly 40 being constrained by the end effector 37. The distal end 68 of the sleeve 62, including the engagement feature 102, is directed through the incision and dilates the tissue as the tool assembly 40 is directed downward towards the pedicle of interest. The distal end 68 of the sleeve 62 may be positioned just above the bone, for example, two to five millimeters above the pedicle of the spine. The locking mechanism 70 is moved from the locked configuration to the unlocked configuration such that the working tool 44 and the dilator 60 are axially movable relative to one another. The dilator 60 may be advanced distally relative to the working tool 44 with sufficient force to cause the engagement feature 102 to engage the bone. In one example, an impacting device such as a mallet 106 and a wrench 108 may be provided in which the wrench 108 is configured to engage the dilator 60 (see FIG. 14), for example, the collar 82 or a head 130 of the dilator 60. The mallet 106 provides an impact force to the wrench 108, which is transferred to the dilator 60 to cause the engagement feature 102 to engage the bone. The engagement of the spikes 104 in the bone helps to minimize skiving of the cutting member 54 of the working tool 44 during cannulation of the pedicle.

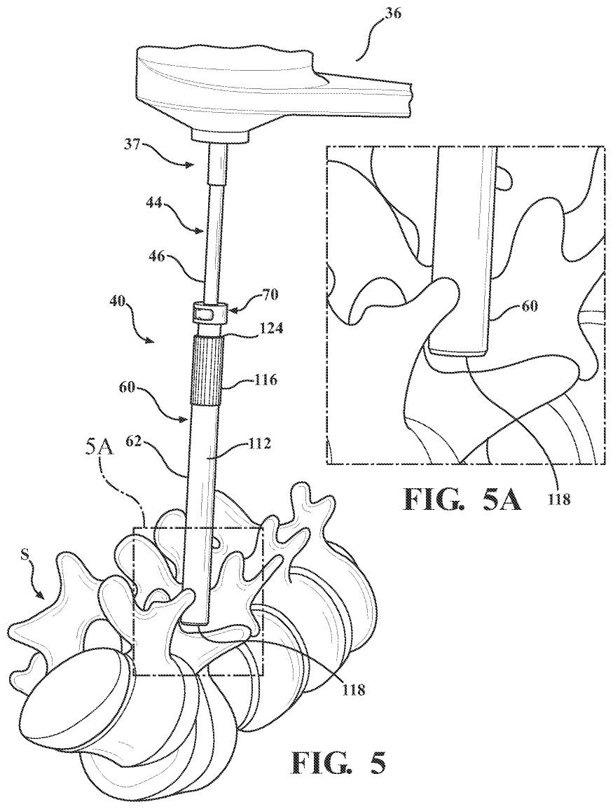

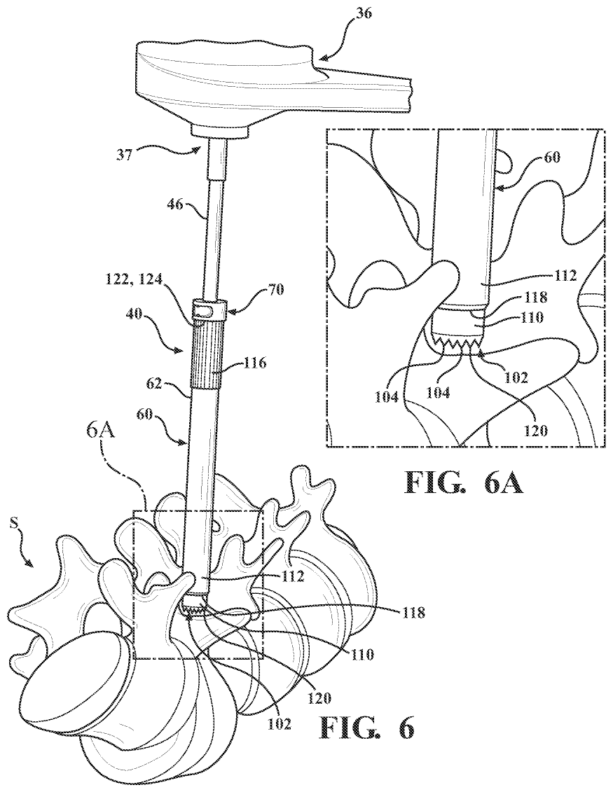

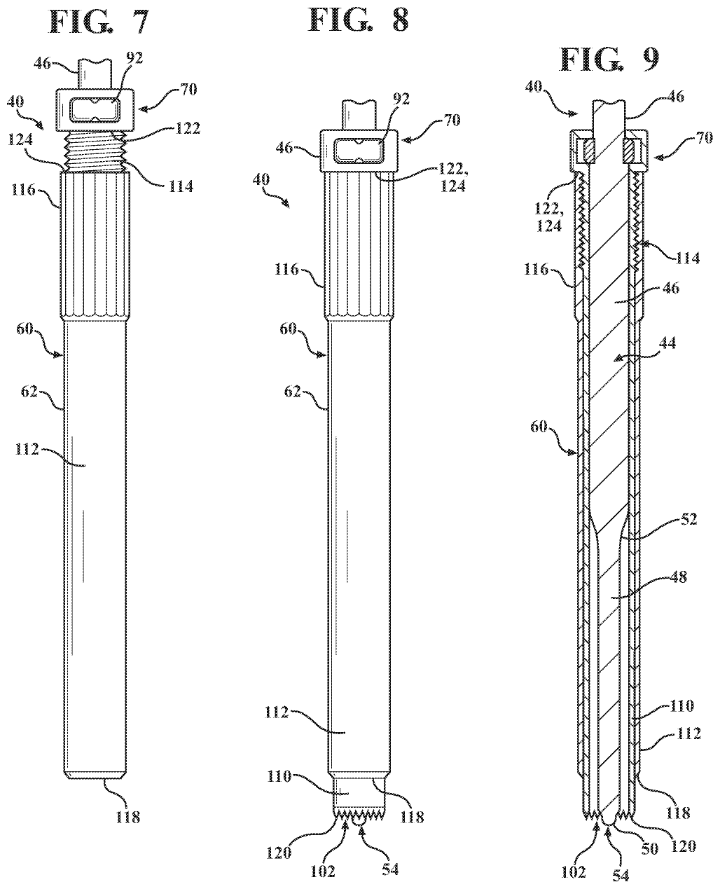

Referring to FIGS. 5-9, the sleeve 62 of the dilator 60 may further include an inner sleeve 110 and an outer sleeve 112. The inner sleeve 110 defines the lumen 64, and the outer sleeve 112 is coaxially and movably disposed over the inner sleeve 110. The implementation of FIGS. 5 and 6 show the outer sleeve 112 slidably disposed over the inner sleeve 110, and the implementation of FIGS. 7-9 show each of the inner and outer sleeves 110, 112 including complementary coupling features 114, for example, external threads on the inner sleeve 110 and internal threads on the outer sleeve 112. Rotation of the outer sleeve 112 relative to the inner sleeve 110 results in axial movement of the outer sleeve 112 relative to the inner sleeve 110. The dilator 60 may include a control surface 116, for example, a grip disposed on the outer sleeve 112. An input may be provided to the control surface 116 to facilitate movement of the outer sleeve 112 relative to the inner sleeve 110, and in particular to expose the engagement feature 102 and/or the inner sleeve 110 beyond a distal end 118 of the outer sleeve 112 in a manner to be described.

The inner sleeve 110 has a length defined between a distal end 120 opposite a proximal end 122, which may correspond to an interface with the collar 82 of the second locking feature 80. The outer sleeve 112 has a length defined between the distal end 118 opposite a proximal end 124. The length of the inner sleeve 110 may be greater than the length of the outer sleeve 112, as best shown in FIG. 9. The tool assembly 40 may be configured to move between an initial configuration in which the engagement feature 102 is recessed within the outer sleeve 112, and a deployed configuration in which the engagement feature 102 extends beyond the distal end 118 of the outer sleeve 112. FIGS. 5 and 5A show the dilator 60 of the tool assembly 40 in the initial configuration. In the initial configuration, the proximal end 124 of the outer sleeve 112 is spaced apart from the proximal end 122 of the inner sleeve 110, and consequently the distal end 120 of the inner sleeve 110 is proximal to or within the distal end 118 of the outer sleeve 112. The engagement feature 102 being recessed within the outer sleeve 112 may avoid possible damage to the overlying tissue from the spikes 104 as the tool assembly 40 is directed through the same. With concurrent reference to FIGS. 6 and 6A, an input is provided to the control surface 116 to slidably move the outer sleeve 112 relative to the inner sleeve 110. The tool assembly 40 assumes the deployed configuration. The proximal end 124 of the outer sleeve 112 may be closer to or abut the proximal end 122 of the inner sleeve 110, and the proximal retraction of the outer sleeve 112 is sufficient to expose the distal end 120 of the inner sleeve 110, including the engagement feature 102, beyond the distal end 118 of the outer sleeve 112. In another implementation, the input may be a twisting input provided to the control surface 116 with engagement of the complementary coupling features 114 causing the proximal retraction of the outer sleeve 112 relative to the inner sleeve 110. For example, FIG. 7 shows the tool assembly 40 in the initial configuration, and FIGS. 8 and 9 show the tool assembly 40 in the deployed configuration. Again, the retraction of the outer sleeve 112 is sufficient to expose the distal end 120 of the inner sleeve 110, including the engagement feature 102, beyond the distal end 118 of the outer sleeve 112. The locking mechanism 70 may be moved from the locked configuration to the unlocked configuration in the manner previously described, and the dilator 60 may be advanced distally relative to the working tool 44 with sufficient force to cause the engagement feature 102 to engage the bone.

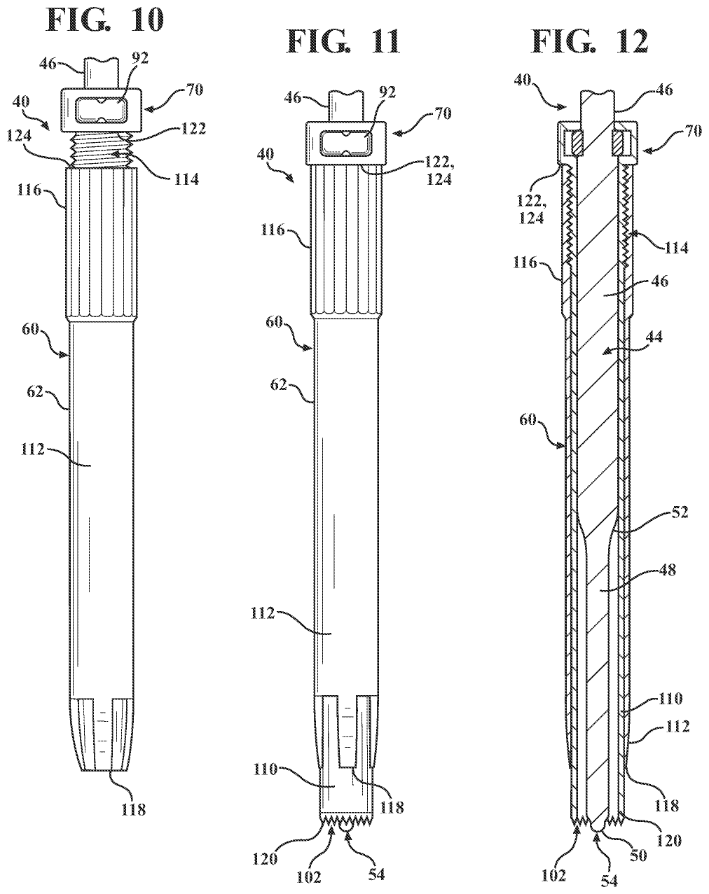

Referring now to FIGS. 10-12, the dilator 60 may include a dilator tip defining the distal end 118 of the dilator 60. The dilator tip may be formed from a resilient material configured to expand over the inner sleeve 110 as the outer sleeve 112 is retracted relative to the inner sleeve 110. The dilator tip may include a taper such that the dilator tip appears substantially frustoconical in shape. The frustoconical shape may facilitate gradual and/or improved dilation of the overlying tissue as the tool assembly 40 is directed through the same. FIG. 10 shows the dilator 60 in the initial configuration, and FIGS. 11 and 12 show the dilator 60 in the deployed configuration. The input may be a twisting input provided to the control surface 116 with engagement of the complementary coupling features 114 causing the proximal retraction of the outer sleeve 112 relative to the inner sleeve 110. As the outer sleeve 112 is retracted proximally, the dilator tip resiliently expands radially outward over the inner sleeve 110. Again, the retraction of the outer sleeve 112 is sufficient to expose the distal end 120 of the inner sleeve 110, including the engagement feature 102, beyond the distal end 118 of the outer sleeve 112.

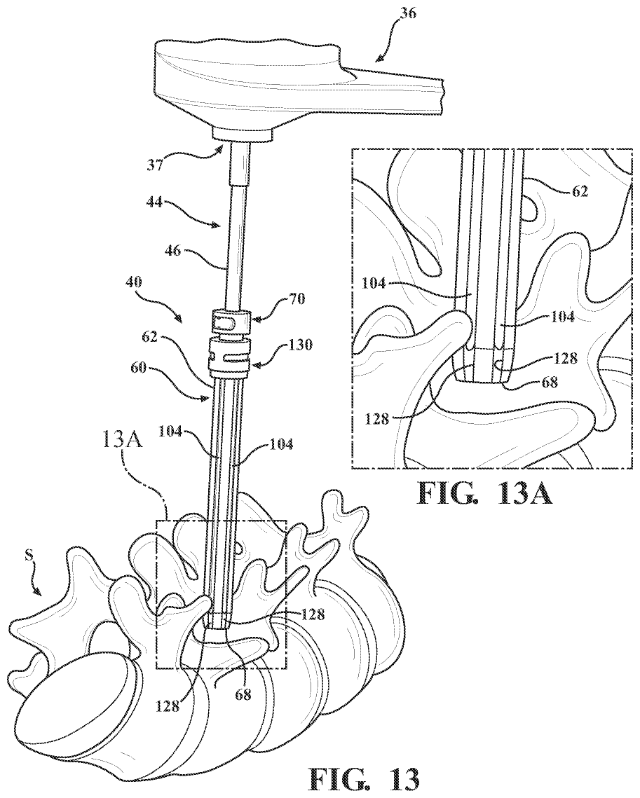

Referring now to FIGS. 13-20, the engagement feature 102 of the dilator 60 may include the spikes 104 movably disposed within grooves 128 defined within the sleeve 62 of the dilator 60. The grooves 128 may extend longitudinally along the sleeve 62 and circumferentially spaced apart from one another. The spikes 104 may include an elongate spike body disposed within the grooves 128 with each of the spikes terminating at a point. The spikes 104 are recessed within the grooves 128 such that the contour of the sleeve 62 is generally uniform. In the initial configuration, the spikes 104 are positioned proximal to the distal end 68 of the sleeve 62 (see FIGS. 13, 13A, 15, 17, 18 and 20), and in the deployed configuration, the spikes 104 are positioned distal to the distal end 68 of the sleeve 62 (see FIGS. 14, 14A, 16 and 19) so as to engage the bone.

The dilator 60 may include the head 130 operably coupled to the spikes 104. With reference to FIG. 14, the head 130 may be an annular protrusion slidably disposed on sleeve 62. The head 130 may be spaced apart from the collar 82 such that the wrench 108 may be inserted therebetween for the impacting device to be used to deploy the spikes 104. Thus, the robotic controller 39 may constrain the end effector 37 as the tool assembly 40 coupled thereto is directed through the overlying tissue and positioned just above the bone, for example, two to five millimeters above the pedicle of the spine. The wrench 108 engages the head 130, and the mallet 106 is used to provide an impact force to the wrench 108. The downward movement of the head 130 moves the spikes 104 distally within the grooves 128. The spikes 104 are moved beyond the distal end 68 of the sleeve 62 such that the tool assembly moves from the initial configuration to the deployed configuration. The locking mechanism 70 is moved from the locked configuration to the unlocked configuration such that the working tool 44 and the dilator 60 are axially movable relative to one another. The dilator 60 may be advanced distally relative to the working tool 44 with sufficient force to cause the engagement feature 102 to engage the bone, as shown in FIG. 13A. The engagement of the spikes 104 in the bone helps to minimize skiving of the cutting member 54 of the working tool 44 during cannulation of the pedicle.

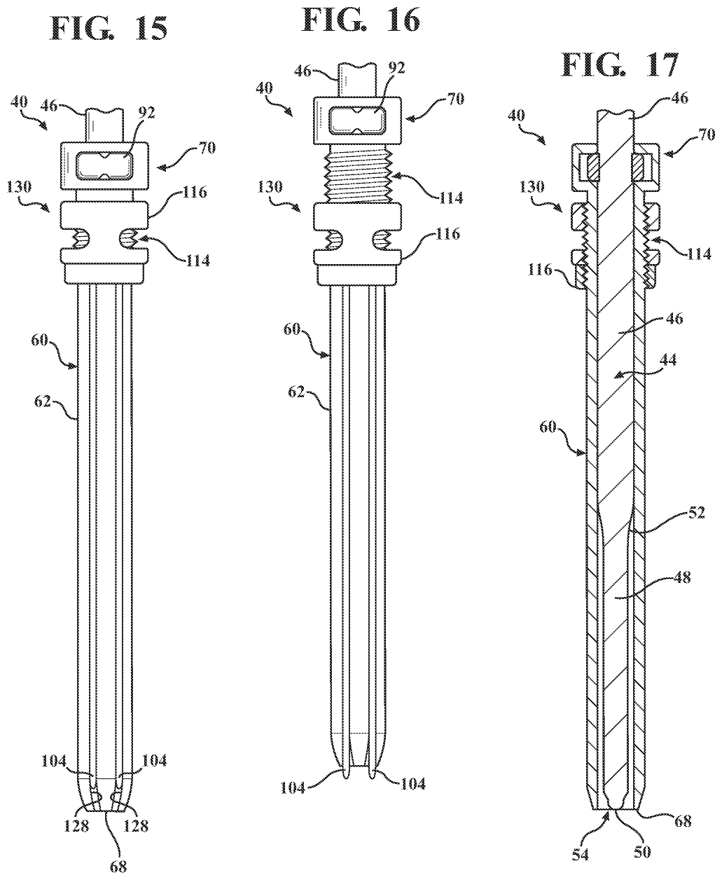

In the implementation of FIGS. 15-17, the input may be a twisting input provided to the control surface 116 with engagement of the complementary coupling features 114 causing the distal advancement of the head 130 and the spikes 104 relative to the sleeve 62. FIGS. 15 and 17 show the tool assembly 40 in the initial configuration, and FIG. 16 shows the tool assembly 40 in the deployed configuration. In another implementation, the twisting input may effectively unlock the head 130 from the sleeve 62, after which the impacting device may be used in the manner previously described to move the tool assembly 40 from the initial configuration to the deployed configuration. In other words, the complementary coupling features 114 may prevent premature axial movement of the head 130 and the spikes 104 relative to the sleeve 62. Once the tool assembly 40 is positioned by the robotic controller 39, the surgeon can provide an input to the control surface 116 until the complementary coupling features 114 disengage. The spikes 104 may have advanced distally during the twisting of the head 130, but not to an extent to extend beyond the distal end 68 of the sleeve 62. Once the complementary coupling features 114 are disengaged, the head 130 is capable of being deployed with the impacting device to penetrate the spikes 104 into the bone.

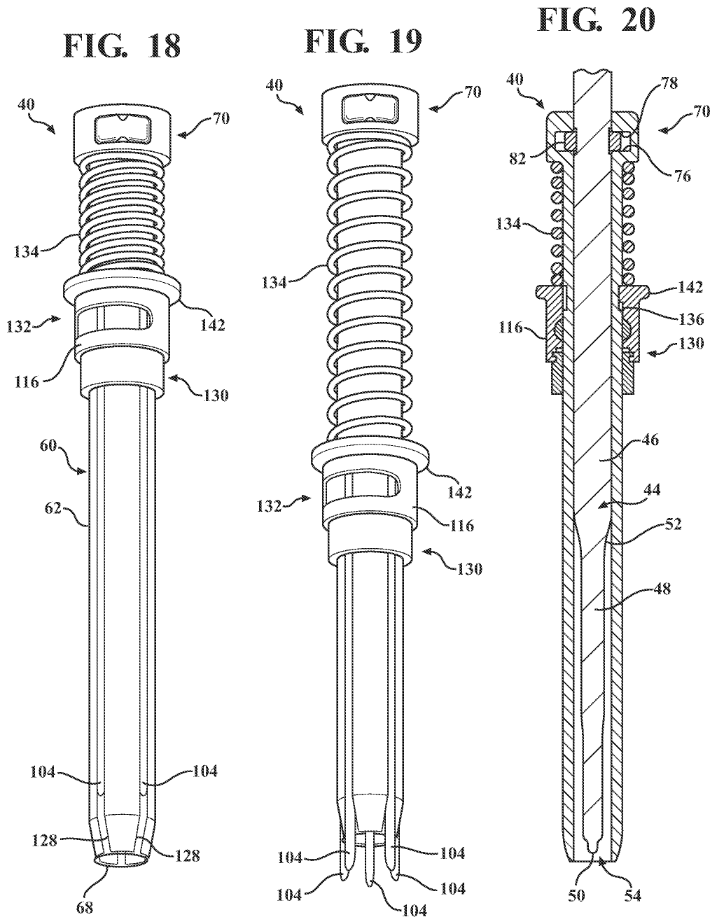

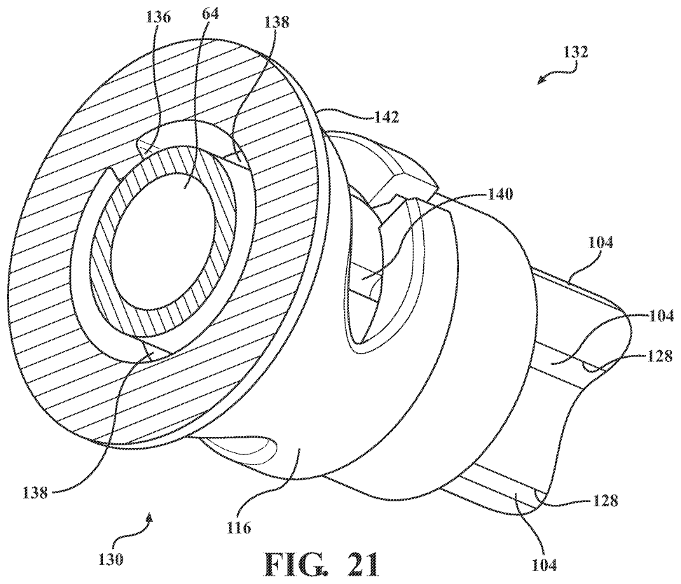

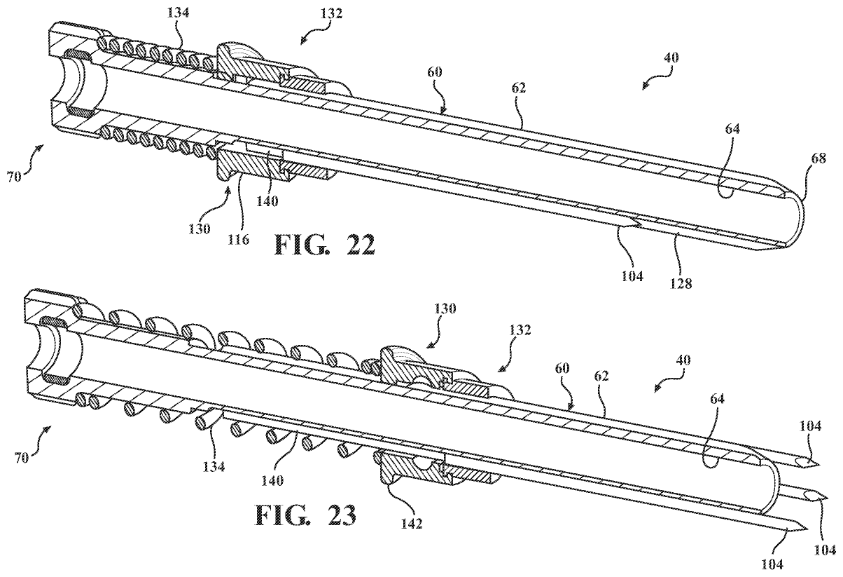

Referring now to FIGS. 18-23, the dilator 60 may include a retention mechanism 132 releasably coupling the sleeve 62 and the spikes 104. The retention mechanism 132 is configured to receive an input to disengage the spikes 104 from the sleeve and permit axial movement of the spikes 104 relative to the sleeve 62. The retention mechanism 132 may include the head 130 previously introduced with modifications to be described to facilitate the releasable engagement, and a biasing element 134 such as a coil spring configured to urge the head 130 and the spikes 104 distally when the retention mechanism 132 is released. The biasing element 134 is shown as being disposed about the sleeve 62 between the collar 82 of the locking mechanism 70 and the head 130. When the retention mechanism 132 is in a locked configuration, the tool assembly 40 is in the initial configuration. The biasing element 134 is held in a biased state with the retention mechanism 132, the spikes 104 are positioned proximal to the distal end 68 of the sleeve 62 and recessed within the grooves 128 (see FIGS. 18, 20 and 22). An input may be provided to the control surface 116 to move the retention mechanism 132 from the locked configuration to an unlocked configuration. The head 130, including the spikes 104 coupled to the head 130, is no longer axially fixed relative to the sleeve 62, and the biasing element 134 releases its stored energy and urges the head 130 and the spikes 104 distally relative to the sleeve 62 (see FIGS. 19 and 21).

FIGS. 20 and 21 shows the retention mechanism 132 including a key 136 coupled to or formed with the head 130. The dilator 60 includes an annular ledge 138 extending about a portion of the sleeve 62, and a slot 140 extending axially through the ledge 138. In the locked configuration, the key 136 is radially positioned out of registration with the slot 140 such that interference between the key 136 and the ledge 138 prevent the biasing element 134 from urging the head 130 distally. To move the retention mechanism 132 from the locked configuration to the unlocked configuration, the input to the control surface 116 may include rotating the head 130 to align the key 136 with the slot 140. The key 136 is free to move within the slot 140 under the influence of the biasing force provided by the biasing element 134. The spikes 104 are moved distally beyond the distal end 68 of the sleeve 62 with sufficient force to penetrate the bone. The tool assembly 40 assumes the deployed configuration. The locking mechanism 70 may be moved from the locked configuration to the unlocked configuration such that the working tool 44 and the dilator 60 are axially movable relative to one another with the dilator 60 engaging the bone.

Should it be desirable to retract the spikes 104 (i.e., move the tool assembly 40 from the deployed configuration to the initial configuration), the head 130 may include a lip 142 configured to facilitate moving the head 130 and the spikes 104 proximally against the biasing force from the biasing element 134. Once the key 136 is positioned proximal to the ledge 138, the user may rotate the head 130 such that the key 136 is out of registration with the slot 140, and interference between the key 136 and the ledge 138 again prevents the biasing element 134 from urging the head 130 distally.

The foregoing description is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A tool assembly for robotic-assisted surgery with a robot, the tool assembly comprising:
a dilator comprising a sleeve defining a lumen;
a working tool comprising a shank configured to be coupled to and supported by the robot, and a cutting member coupled to the shank, the working tool sized to be coaxially movable within the lumen of the dilator; and
a locking mechanism comprising a first locking feature coupled to the shank, and a second locking feature coupled to the sleeve, wherein the first and second locking features are configured to releasably engage one another to prevent axial movement of the working tool relative to the dilator such that the working tool supports the dilator when the working tool is coupled to the robot, wherein the locking mechanism further comprises an actuator configured to receive an input to disengage the first and second locking features and permit axial movement of the working tool within the lumen of the dilator.

2. The tool assembly of claim 1, wherein the dilator further comprises at least one spike coupled to the sleeve and configured to penetrate bone.

3. The tool assembly of claim 1, wherein the sleeve is an inner sleeve defining the lumen, the dilator further comprising an outer sleeve coaxially disposed over the inner sleeve, wherein the inner sleeve is movably coupled to the outer sleeve.

4. The tool assembly of claim 3, wherein the inner sleeve has an inner sleeve length defined between the second locking feature and a distal end of the inner sleeve, and the outer sleeve has an outer sleeve length defined between opposing proximal and distal ends of the outer sleeve, wherein the inner sleeve length is greater than the outer sleeve length; and
wherein the working tool has a tool length configured to align the cutting member to the distal end of the inner sleeve when the first and second locking features are engaged.

5. (canceled)

6. The tool assembly of claim 3, wherein the outer sleeve further comprises a dilator tip defining a distal end of the outer sleeve, wherein the dilator tip is at least partially formed from a resilient material configured to expand over the inner sleeve as the outer sleeve is retracted relative to the inner sleeve.

7. The tool assembly of claim 2, wherein the at least one spike is movably coupled to the sleeve and configured to be selectively extended beyond a distal end of the sleeve to penetrate the bone.

8. The tool assembly of claim 7, wherein the at least one spike further comprises an elongate spike body slidably disposed within a groove defined by the sleeve.

9. The tool assembly of claim 7, wherein the dilator further comprises a biasing element operably coupled to the at least one spike and configured to selectively extend the at least one spike beyond the distal end of the sleeve to penetrate the bone;
wherein the dilator further comprises a retention mechanism releasably coupling the sleeve and the at least one spike, wherein the retention mechanism is configured to receive an input to permit movement of the at least one spike relative to the sleeve; and
wherein the biasing element is a coil spring disposed about the sleeve between the locking mechanism and the retention mechanism.

10. (canceled)

11. (canceled)

12. The tool assembly of claim 1, wherein the first locking feature comprises a necked portion of the shank defining two stepped surfaces, and the second locking feature comprises a biasing element coupled to the actuator and configured to urge the actuator into releasable engagement with one of the stepped surfaces.

13. A method for performing robotic-assisted surgery with a robot, a tool assembly including a dilator, a working tool, and a locking mechanism releasably coupling the dilator and the working tool, the method comprising the steps of:
with the working tool coaxially disposed within the dilator and with the locking mechanism in a locked configuration in which axial movement of the working tool relative to the dilator is prevented, coupling the working tool to the robot such that the robot supports the working tool and the dilator;
operating the robot to advance a distal end of the working tool and a distal end of the dilator into an incision in overlying tissue and above or in engagement with a bone;
actuating the locking mechanism from the locked configuration to an unlocked configuration in which axial movement of the working tool relative to the dilator is permitted; and
operating the robot to advance the distal end of the working tool and to resect the bone, wherein a position of the dilator is maintained by the overlying tissue supporting the dilator.

14. The method of claim 13, wherein the dilator includes at least one spike, the method further comprising facilitating the engagement of the at least one spike with the bone.

15. The method of claim 14, wherein the step of facilitating the engagement of the at least one spike with the bone is after the step of actuating the locking mechanism from the locked configuration to the unlocked configuration.

16. The method of claim 14, wherein the dilator further includes an inner sleeve including the at least one spike, and an outer sleeve coaxially disposed over the inner sleeve, the method further comprising retracting the outer sleeve relative to the inner sleeve to expose the at least one spike; and
wherein the inner sleeve and the outer sleeve include complementary threads, the method further comprising providing a rotational input to the outer sleeve to retract the outer sleeve relative to the inner sleeve to expose the at least one spike.

17. (canceled)

18. The method of claim 14, wherein the dilator comprises a sleeve, wherein the sleeve defines a groove and the at least one spike is disposed within the groove, the method further comprising advancing the at least one spike beyond a distal end of the sleeve.

19. The method of claim 18, wherein the dilator further includes a collar coupled to the at least one spike, the method further comprising impacting the collar with an impacting device to advance the at least one spike beyond the distal end of the sleeve.

20. The method of claim 18, wherein the tool assembly further includes a retention mechanism releasably coupling the at least one spike and the dilator, the method further comprising providing an input to the retention mechanism to disengage the at least one spike from the dilator.

21. (canceled)

22. A tool assembly for robotic-assisted surgery with a robot, the tool assembly comprising:
a dilator comprising an inner sleeve defining a lumen and comprising at least one spike, the dilator further comprising an outer sleeve coaxially disposed over the inner sleeve, wherein at least one of the inner sleeve and the outer sleeve is movable from an initial configuration in which the at least one spike is recessed within the outer sleeve, and a deployed configuration in which the at least one spike extends beyond the outer sleeve for penetrating bone;
a working tool comprising a shank configured to be coupled to and supported by the robot, and a cutting member coupled to the shank, the working tool sized to be slidably and coaxially movable within the lumen of the dilator; and
a locking mechanism releasably coupling the working tool and the dilator.

23. The tool assembly of claim 22, wherein the locking mechanism further comprises a necked portion of the shank defining two stepped surfaces, and an actuator positioned for releasable engagement with one of the stepped surfaces.

24. The tool assembly of claim 22, wherein the inner sleeve has an inner sleeve length defined between opposing proximal and distal ends of the inner sleeve, and the outer sleeve has an outer sleeve length defined between opposing proximal and distal ends of the outer sleeve, wherein the inner sleeve length is greater than the outer sleeve length.

25. The tool assembly of claim 22, wherein the outer sleeve further comprises a dilator tip defining a distal end of the outer sleeve, wherein the dilator tip is at least partially formed from resilient materials configured to expand over the inner sleeve as the outer sleeve is moved from the initial configuration to the deployed configuration.
